(19) United States
(12) Patent Application Publication
(10) Pub. No.: US 2010/0234400 A1
(43) Pub. Date: Sep. 16, 2010

(54) COMPOUNDS THAT MAINTAIN PLURIPOTENCY OF EMBRYONIC STEM CELLS

(75) Inventors: Shuibing Chen, San Diego, CA (US); Sheng Ding, San Diego, CA (US); Feng Yan, La Jolla, CA (US); Peter G. Schultz, La Jolla, CA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton (BM); The Scripps Research Institute, La Jolla, CA (US)

(21) Appl. No.: 11/917,138

(22) PCT Filed: Jun. 8, 2006
(86) PCT No.: PCT/US06/22648

§ 371 Date: May 7, 2010

Related U.S. Application Data
(60) Provisional application No. 60/689,359, filed on Jun. 10, 2005.

(51) Int. Cl. A61K 31/519; C12N 5/071

(52) U.S. Cl. 514/262.1; 435/366

(57) ABSTRACT

The present invention relates to methods and compositions for culturing embryonic stem (ES) cells. The methods relate to growing the ES cells in the presence of small molecules of formula (I) that maintain the pluripotency/self-renewal of the cells without feeder cells and LIF in serum-free conditions. These methods in part facilitate much more consistency in embryonic stem cell production, providing, for example, new avenues in the practical applications of embryonic stem cells in regenerative medicine.

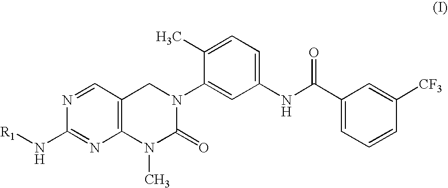

### CROSS-REFERENCE TO RELATED APPLICATIONS

This application claims the benefit of priority to U.S. Provisional Patent Application No. 60/689,359, filed 10 Jun. 2005. The full disclosure of this application is incorporated herein by reference in its entirety and for all purposes.

### BACKGROUND OF THE INVENTION

1. Field of the Invention

The present invention relates to methods and compositions for culturing embryonic stem (ES) cells. The methods relate to growing the ES cells in the presence of small molecules that maintain the pluripotency/self-renewal of the cells without feeder cells and LIF in serum-free conditions. These methods in part facilitate much more consistency in embryonic stem cell production, providing, for example, new avenues in the practical applications of embryonic stem cells in regenerative medicine.

2. Background

Embryonic stem cells are difficult to maintain in culture because they tend to spontaneously differentiate (i.e., acquire specialized structural and/or functional features). Stem cells differentiate as a result of many factors, including growth factors, extracellular matrix molecules and components, environmental stressors and direct cell-to-cell interactions.

Generating cultures of mouse or human embryonic stem cells that remain in a proliferating, undifferentiated state is a multistep process that includes growing the cells in growth medium supplemented with fetal calf serum and sometimes on a “feeder” layer of non-dividing cells. The mouse embryonic stem cells can be grown in vitro without feeder cells if the cytokine leukemia inhibitory factor (LIF) is added to the culture medium but this is only effective at moderate to high cell densities and colony formation from single cells requires the presence of either serum or a feeder layer. Furthermore, for human embryonic stem cells, even in the presence of serum, LIF is not adequate to support self-renewal.

The present invention provides a method of using small molecules for self-renewal of embryonic stem cells in serum-free culture conditions without the use of LIF. Using small molecules of the invention to maintain pluripotency of embryonic stem cells allows for much more consistency in embryonic stem cell production, providing, for example, new avenues in the practical applications of embryonic stem cells in regenerative medicine.

### SUMMARY OF THE INVENTION

In one aspect, the present invention provides a method of maintaining pluripotent stem cells, comprising the steps of growing the cells in: a) a basal medium; and b) a compound of Formula I:

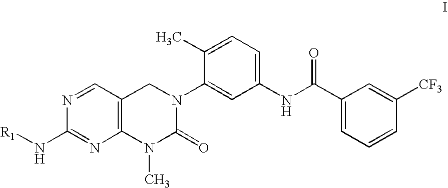

in which:

R₁ is selected from hydrogen, C₁₋₆alkyl, C₂₋₆alkenyl, C₆₋₁₀aryl-C₀₋₄alkyl, C₅₋₁₀heteroaryl-C₀₋₄alkyl, C₃₋₁₀cycloalkyl-C₀₋₄alkyl and C₃₋₄₀heterocycloalkyl-C₀₋₄alkyl; wherein any alkyl or alkenyl of R₁ is optionally substituted by one to three radicals independently selected from halo, hydroxy, C₁₋₆alkyl and —NR₂R₃; wherein any aryl, heteroaryl, cycloalkyl or heterocycloalkyl of R₁ is optionally substituted by one to three radicals selected from halo, hydroxy, cyano, C₁₋₆alkyl, C₁₋₆alkoxy, C₂₋₆alkenyl, halo-substituted-alkyl, halo-substituted-alkoxy, —XNR₂R₃, —XOXNR₂R₃, —XNR₂S(O)₀₋₂R₃, —XC(O)NR₂R₃, —XNR₂C(O)XOR₂, —XNR₂C(O)NR₂R₃, —XNR₂XNR₂R₃, —XC(O)NR₂XNR₂R₃, —XNR₂XOR₂, —XOR₂, —XNR₂C(═NR₂)NR₂R₃, —XS(O)₀₋₂R₄, —XNR₂C(O)R₂, —XNR₂C(O)XNR₂R₃, —XNR₂C(O)R₄, —XC(O)R₄, —XR₄, —XC(O)OR₃ and —XS(O)₀₋₂NR₂R₃; wherein X is a bond or C₁₋₄alkylene; R₂ and R₃ are independently selected from hydrogen, C₁₋₆alkyl and C₃₋₁₂cycloalkyl; and R₄ is C₃₋₁₀heterocycloalkyl optionally substituted with 1 to 3 radicals selected from C₁₋₆alkyl, —XNR₂R₃, —XNR₂XNR₂R₂, XNR₂XOR₂ and —XOR₂; wherein X, R₂ and R₃ are as described above; and the N-oxide derivatives, prodrug derivatives, protected derivatives, individual isomers and mixture of isomers thereof; and the pharmaceutically acceptable salts and solvates (e.g. hydrates) of such compounds.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group. “Heteroaryl” is as defined for aryl where one or more of the ring members are a heteroatom. For example heteroaryl includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc. “Heterocycloalkyl” means cycloalkyl, as defined in this application, provided that one or more of the ring carbons indicated, are replaced by a moiety selected

from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, piperazinyl, piperidinyl, piperidinylone, 2-Oxo-pyrrolidin-1-yl, 1,4-dioxa-8-aza-spiro[4.5]dec-8-yl, etc.

“Halogen” (or halo) preferably represents chloro or fluoro, but may also be bromo or iodo.

“Treat”, “treating” and “treatment” refer to a method of alleviating or abating a disease and/or its attendant symptoms.

### DESCRIPTION OF THE PREFERRED EMBODIMENTS

The present invention relates to methods and compositions for culturing ES cells. The methods relate to growing the ES cells in the presence of small molecules that maintain the pluripotency/self-renewal of the cells without feeder cells and LIF in serum-free conditions.

In one embodiment, with reference to compounds of Formula I:

R₁ is selected from hydrogen, C₁₋₆alkyl, C₂₋₆alkenyl, C₆₋₁₀aryl-C₀₋₄alkyl, C₅₋₁₀heteroaryl-C₀₋₄alkyl, C₃₋₁₀cycloalkyl-C₀₋₄alkyl and C₃₋₁₀heterocycloalkyl-C₀₋₄alkyl; wherein any alkyl or alkenyl of R₁ is optionally substituted by one to three radicals independently selected from halo, hydroxy, C₁₋₆alkyl and —NR₂R₃; wherein any aryl, heteroaryl, cycloalkyl or heterocycloalkyl of R₁ is optionally substituted by one to three radicals selected from halo, hydroxy, cyano, C₁₋₆alkyl, C₁₋₆alkoxy, C₂₋₆alkenyl, halo-substituted-alkyl, halo-substituted-alkoxy, —XNR₂R₃, —XOXNR₂R₃, —XNR₂S(O)₀₋₂R₃, —XC(O)NR₂R₃, —XNR₂C(O)XOR₂, —XNR₂C(O)NR₂R₃, —XNR₂XNR₂R₃, —XC(O)NR₂XNR₂R₃, —XNR₂XOR₂, XOR₂, —XNR₂C(═NR₂)NR₂R₃, —XS(O)₀₋₂R₄, —XNR₂C(O)R₂, —XNR₂C(O)XNR₂R₃, —XNR₂C(O)R₄, —XC(O)R₄, —XR₄, —XC(O)OR₃ and —XS(O)₀₋₂NR₂R₃; wherein X is a bond or C₁₋₄alkylene; R₂ and R₃ are independently selected from hydrogen, C₁₋₆alkyl and C₃₋₁₂cycloalkyl; and R₄ is C₃₋₁₀heterocycloalkyl optionally substituted with 1 to 3 radicals selected from C₁₋₆alkyl, —XNR₂R₃, —XNR₂XNR₂R₂, XNR₂XOR₂ and —XOR₂; wherein X, R₂ and R₃ are as described above.

In another embodiment, R₁ is selected from hydrogen, methyl, ethyl, isopropyl, cyclopropyl, cyclobutyl, cyclopentyl, cyclohexyl, pyrimidinyl, 3-hydroxy-1-methyl-propyl hydroxy-ethyl, phenyl, morpholino, benzyl, [1,2,4]triazol-4-yl, allyl, 2-methyl-allyl, 2-(2-oxo-pyrrolidin-1-yl)-ethyl, piperazinyl-ethyl, piperazinyl-propyl, thiazolyl, oxazolyl, pyridinyl, pyrazolyl, piperidinyl, thiazolyl, ethyl-pyrrolidinyl-methyl, morpholino-propyl, dimethyl-amino-propyl, diethyl-amino-propyl, diethyl-amino-butyl, ethoxy-carbonyl-methyl and [1,2,4]triazin-3-yl, [1,3,4]thiadiazolyl; wherein any aryl, heteroaryl, cycloalkyl or heterocycloalkyl is optionally substituted with 1 to 3 radicals independently selected from methyl, ethyl, cyano, hydroxy, methoxy, amino-carbonyl-amino, hydroxy-methyl, methyl-piperazinyl, methyl-piperazinyl-carbonyl, ethyl-piperazinyl, methyl-piperazinyl-methyl, morpholino-sulfonyl, methyl-piperazinyl-sulfonyl, methyl-piperazinyl-carbonyl-amino, methyl-sulfonyl-amino, amino-carbonyl, amino-sulfonyl, hydroxy-ethyl, hydroxy-methyl-carbonyl-amino, formyl-amino, dimethyl-amino, dimethyl-amino-methyl, dimethyl-amino-ethyl, isopropyl-amino-ethyl, carboxy, amino-ethyl-amino, methyl-amino-ethyl, morpholino-ethyl, morpholino-methyl, amino-ethyl, imidazolyl-propyl, piperazinyl-ethyl, piperazinyl, trifluoromethyl, diethyl-amino-ethyl, fluoro, morpholino, dimethyl-amino-ethyl-amino-carbonyl, diethyl-amino-ethoxy, 2-amino-propionylamino, dimethyl-amino-pyrrolidinyl, (2-dimethylamino-ethyl)-methyl-amino, 2-dimethylamino-1-methyl-ethoxy and diethyl-amino.

Preferred compounds of the invention are selected from: N-{3-[7-(2-Ethyl-2H-pyrazol-3-ylamino)-1-methyl-2-oxo-1,4-dihydro-2H-pyrimido[4,5-d]pyrimidin-3-yl]-4-methyl-phenyl}-3-trifluoromethyl-benzamide; N-{4-Methyl-3-[1-methyl-7-(2-methyl-2H-pyrazol-3-ylamino)-2-oxo-1,4-dihydro-2H-pyrimido[4,5-d]pyrimidin-3-A-phenyl}-3-trifluoromethyl-benzamide; N-{3-[7-(2,6-Dimethyl-pyridin-4-ylamino)-1-methyl-2-oxo-1,4-dihydro-2H-pyrimido[4,5-d]pyrimidin-3-yl]-4-methyl-phenyl}-3-trifluoromethyl-benzamide; N-{3-[7-(3-Hydroxy-phenylamino)-1-methyl-2-oxo-1,4-dihydro-2H-pyrimido[4,5-d]pyrimidin-3-yl]-4-methyl-phenyl]-3-trifluoromethyl-benzamide; N-{3-[7-(2,5-Dimethyl-2H-pyrazol-3-ylamino)-1-methyl-2-oxo-1,4-dihydro-2H-pyrimido[4,5-d]pyrimidin-3-yl]-4-methyl-phenyl]-3-trifluoromethyl-benzamide; N-{3-[7-(3-Amino-phenylamino)-1-methyl-2-oxo-1,4-dihydro-2H-pyrimido[4,5-d]pyrimidin-3-yl]-4-methyl-phenyl}-3-trifluoromethyl-benzamide; N-{3-[7-(3-Methanesulfonylamino-phenylamino)-1-methyl-2-oxo-4,4-dihydro-2H-pyrimido[4,5-d]pyrimidin-3-yl]-4-methyl-phenyl}-3-trifluoromethyl-benzamide; N-[4-Methyl-3-(1-methyl-7-methylamino-2-oxo-1,4-dihydro-2H-pyrimido[4,5-d]pyrimidin-3-yl)-phenyl]-3-trifluoromethyl-benzamide; and N-[3-(7-Ethylamino-1-methyl-2-oxo-1,4-dihydro-2H-pyrimido[4,5-d]pyrimidin-3-yl)-4-methyl-phenyl]-3-trifluoromethyl-benzamide.

Additional preferred compounds of Formula I are detailed in the Examples and Table I, infra.

### Utility

ES cells are derived from pre-implantation embryos and retain the developmental potency of fetal founder cells, being able to generate cell and tissue types of all three germ layers in vitro and in vivo. ES cells can be viewed as cells that must choose between self-renewal (pluripotency) or alternative fates of differentiation at each division. The signals that govern the choice of differentiation path are provided by growth factors in the cells microenvironment. Growth factors can be available in serum or can be produced by feeder cells.

Identifying these growth factors and defining their respective inputs are critical to understanding the developmental and physiological regulation of stem cell-mediated tissue generation, turnover, and repair. Furthermore, extending such knowledge to control the expansion and differentiation of stem cells ex vivo holds promise for applications in regenerative medicine and biopharmaceutical discovery.

Mouse ES cells were originally isolated and maintained by co-culture on a feeder layer of mitotically inactivated mouse embryo fibroblasts. The essential function of the fibroblast feeder layer is to provide the cytokine leukemia inhibitory factor (LIF). LIF null fibroblasts are deficient at supporting self-renewal and LIP can replace the requirement for feeders in both routine propagation and de novo derivation of mouse ES cells. LIF and related cytokines that engage the gp 130 receptor provide the only molecularly defined pathway that will sustain long-term self-renewal of mouse ES cells with of the cardinal attributes of undifferentiated phenotype, pluripotency and embryo colonization capacity.

ES cells can be propagated in a commercial serum substitute supplemented with LIF, but this is only effective at moderate to high cell densities and colony formation from single cells requires the presence of either serum or a feeder layer. Furthermore, for human ES cells, even in the presence of serum, LIF is not adequate to support self-renewal.

The methods of the present invention allow for the maintenance of pluripotent stem cells without feeder cells and LIP in serum-free conditions. Compounds of the invention effect self-renewal of mES cells via their interaction with ERK1 and RasGAP. For example, sustained ERK1/2 activation leads to neuronal differentiation while inhibiting RasGAP may activate signaling by Ras or Ras-like GTPases, which in turn can enhance self-renewal through P13K or other signaling pathways.

Bone morphogenic proteins (BMPs) have been implicated as the factor contained in serum or provided by feeder layers that acts in concert with LIF to maintain undifferentiated mouse ES cells in vitro. It has been suggested that BMPs can replace serum and feeder cell requirements in ES cell culture by activating the Smad pathway and inducing expression of the Id gene, a common target of Smad signaling that appears to block differentiation by negatively regulating basic helix-loop-helix proteins. Although the exact mechanism by which BMP promotes self-renewal of ES cells is not certain, recent work suggests that it might also inhibit the mitogen-activated protein kinase (MAPK) pathway independent of Smads. Importantly, inhibition of p38 MAPK facilitates derivation of ES cells from blastocysts lacking Alk-3 (BMPRIA), and ES cells can be derived from blastocysts lacking Smad4 (the common partner of all Smads), supporting the hypothesis that BMP acts by means of different mechanisms depending on the presence or absence of serum and feeders.

Considering the possibility that serum and feeder cells provide cell survival signals manifest as growth factors and cytokines and that extrinsic survival signals are especially critical in low cell density conditions, where stimulation through autocrine and paracrine factors are minimal, ES cells likely become apoptotic in suboptimal culture conditions (i.e., in the absence of serum and feeder cells). At low cell density, ES cells infrequently generate pluripotent colonies. To analyze the effect of single cytokines, growth factors, and other molecules on the self-renewal and differentiation of ES cells, it would be optimal if cells could be protected from apoptotic cell death in serum-free and feeder-free conditions. Although the use of N2- and B27-supplemented media to expand ES cells in serum-free and feeder-free conditions improves viability and, thus, allows their survival even at low cell density conditions, LIF plus these supplements cannot support the self-renewal of ES cells unless the culture is further supplemented with BMP. Because N2 and B27 supplements contain hormones (corticosterone, progesterone, and T3) and retinyl acetate (a precursor of retinoic acid) and some of these components are used in ES cell differentiation protocols, their presence complicates the analysis of the effects of single cytokines, growth factors, and other molecules on the self-renewal and differentiation of ES cells.

Consequently, the development of small molecules for self-renewal of ES cells in serum-free culture conditions, such as described by the present invention, will allow much more consistency in ES cell production, providing new avenues in practical application of ES cells in research and in regenerative medicine.

Further, development of small molecules for self-renewal of ES cells in serum-free culture conditions, such as described by the present invention, is essential for delimiting the ES cell culture environment and thereby allowing for the definition and control of signaling inputs that direct self-renewal or differentiation.

The mechanism of pluripotency may also contribute to our understanding of tumorigenesis (pluripotent stem cells can form tumors in vivo, and molecular alterations in the “sternness” genes may also lead to tumors). In addition, there is a growing body of evidence suggesting a close relationship between stem cells and tumor cells: the self-renewal mechanisms of normal stem cells and tumor cells are similar; deregulation of developmental signaling pathways involved in stem cell self-renewal is associated with oncogenesis; tumors contain “cancer stem cells” which may arise from normal stern cells.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I can be prepared by proceeding as in the following Reaction Scheme I:

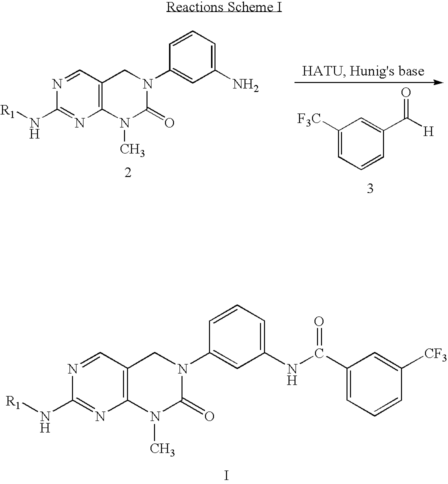

in which R₁ is as defined for Formula I in the Summary of the Invention.

Compounds of Formula I can be prepared by coupling compounds of formula 2 with compounds of formula 3 using a suitable acyl activating reagent (e.g., HATU) in the presence of a suitable base (e.g., DIEA, or the like) and an appropriate solvent (e.g., DMF) and can take up to 3 hours to complete.

Compounds of Formula I can be prepared by proceeding as in the following Reaction Scheme II:

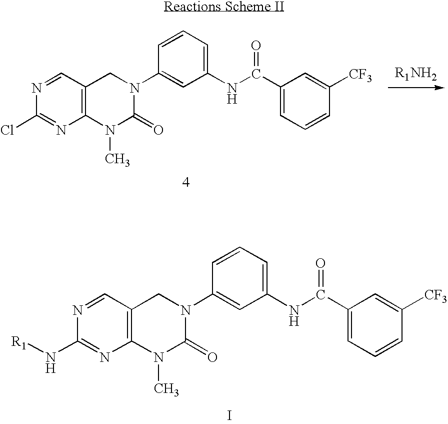

in which R₁ is as defined for Formula I in the Summary of the Invention.

A compound of Formula I can be prepared by reacting a compound of formula 4 with a suitable amine in the absence or presence of an appropriate solvent (e.g., AcOH-water). A compound of Formula I can be also prepared by reacting a compound of formula 4 with a suitable amine in the presence of a suitable solvent (e.g., 1-butanol) with the aid of p-toluenesulfonic acid at elevated temperatures.

Alternatively, a compound of Formula I can be prepared by reacting a compound of formula 4 with a compound of formula R₁H by three methods. For the heteroaryl amine or aryl amine, the reaction proceeds in the presence of a suitable catalyst (e.g., Pd (II) salt, or the like) and a suitable solvent (e.g., 1,4-dioxane, or the like), in a temperature range of about 80 to about 150° C. and can take up to about 20 hours to complete. The reaction conditions for alkyl amine displacement involves heating a compound of formula 4 with 5-10 equivalents of amine in a suitable solvent (e.g. DMSO, DMF, or the like). For condensations of formula 4 with aryl amine, these are carried out in the presence of acid (e.g., TsOH, HOAc, HCl, or the like) in a suitable solvent (e.g., DMSO, DMF, alcohol or the like).

Detailed examples of the synthesis of a compound of Formula I can be found in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques; Andre Collet, Samuel H. Wilen, “Enantiomers, Racemates and Resolutions”, John Wiley And Sons, Inc., 1981.

In summary, the compounds of Formula I can be made by a process, which involves:

(a) those of reaction schemes I and II; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following examples that illustrate the preparation of compounds of Formula I (Examples) according to the invention.

### Example 1

### N-{3-[7-(3-Amino-phenylamino)-1-methyl-2-oxo-1,4-dihydro-2H-pyrimido[4,5-d]pyrimidin-3-yl]-4-methyl-phenyl}-3-trifluoromethyl-benzamide

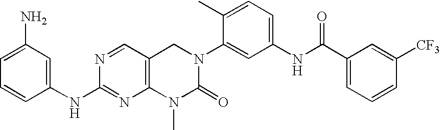

5-Bromo-2,4-dichloro-pyrimidine (2.41 g, 10.6 mmol) is slowly treated with methylamine (8 M in EtOH, 3.3 mL) in THF (15 mL) at about −20° C. After stirring for 30 minutes at about −20° C., the reaction mixture is partitioned between CHCl₃ and saturated NaHCO₃. The aqueous layer is extracted with additional CHCl₃ twice and the combined organic layer is dried over MgSO₄, filtered and concentrated. The crude product is purified by column chromatography (SiO₂, EtOAc/Hexane=3/7) to give 1.76 g (75%) of (5-bromo-2-chloro-pyrimidin-4-yl)-methylamine as a white solid.

A mixture of (5-bromo-2-chloro-pyrimidin-4-yl)-methylamine (3.75 g, 16.9 mmol), tris(dibenzylidineacetone)dipalladium(0) (388 mg, 0.4 mmol), and tri-2-furylphosphine (777 mg, 3.3 mmol) in DMF is stirred for 20 minutes at room temperature and then tributylvinyltin (5.93 mL, 20.3 mmol) is added. After stirring for 16 hours at about 65° C., the reaction mixture is cooled to room temperature and stirred with a 10% aqueous solution of potassium fluoride (800 mL) and diethyl ether (600 mL) for 1 hour before filtering through a pad of Celite. The pad of Celite is rinsed with a further portion of diethyl ether (200 mL). The aqueous layer is separated and extracted with CHCl₃. The combined organic extract is dried over MgSO₄ and concentrated under reduced pressure to give crude oil which is purified by flash column chromatography (SiO₂, EtOAc/Hx=1/4) to afford (2-chloro-5-vinyl-pyrimidin-4-yl)-methylamine (2.63 g, 92%) as a white solid.

A solution of (2-chloro-5-vinyl-pyrimidin-4-yl)-methylamine (2.50 g, 14.7 mmol) in CHCl₃/MeOH (15 mL/15 mL) is bubbled by ozone for 30 minutes and then passed by a stream of argon for 3 minutes at −78° C. The reaction mixture is allowed to warm up to room temperature and treated with dimethyl sulfide (3.24 mL, 44.1 mmol). The reaction mixture is concentrated under reduced pressure to give colorless oil that is purified by flash column chromatography (SiO₂, EtOAc/Hx=1/3) over silica gel to give 2-chloro-4-methylamino-pyrimidine-5-carbaldehyde (2.40 g, 95%) as a white solid.

A solution of 2-chloro-4-methylamino-pyrimidine-5-carbaldehyde (1.08 g, 6.3 mmol) and N-(3-amino-4-methyl-phenyl)-3-trifluoromethylbenzamide (2.04 g, 6.9 mmol) in MeOH (70 mL) is stirred for 2 hours at 45° C. and then treated with sodium cyanoborohydride (1.19 g, 18.9 mmol) and acetic acid (1 mL) sequentially. After stirring for 2 hours at room temperature, the reaction mixture is diluted with CHCl₃ and washed with saturated NaHCO₃. The organic layer is dried over MgSO₄ and concentrated under reduced pressure. The residue is purified by flash column chromatography (SiO₂, EtOAc/hexane=1/2) to give N-{3-[(2-chloro-4-methylaminopyrimidin-5-ylmethyl)amino]-4-methylphenyl}-3-trifluoromethylbenzamide (1.80 g, 64%) as a white solid.

To a stirred solution of N-{3-[(2-chloro-4-methylaminopyrimidin-5-ylmethyl)amino]-4-methylphenyl}-3-trifluoromethylbenzamide (559 mg, 1.24 mmol) and triethylamine (693 μL, 4.97 mmol) in THF (15 mL) is added triphosgene (147 mg, 0.49 mmol) in THF (5 mL) at 0° C., and the mixture is stirred for 30 minutes at room temperature. The precipitate is filtered off and the filtrate is stirred for 3 hours at 110° C. The reaction mixture is then diluted with EtOAc and washed with saturated NaHCO₃. The organic layer is dried over MgSO₄ and concentrated under reduced pressure to give crude oil which is purified by flash column chromatography (SiO₂, EtOAc/hexane=1/2) to give N-[3-(7-chloro-2-oxo-1,4-dihydro-2H-pyrimido[4,5-d]pyrimidin-3-yl)-4-methylphenyl]-3-trifluoromethylbenzamide (420 mg, 71%) as a white solid.

A mixture of N-[3-(7-chloro-2-oxo-1,4-dihydro-2H-pyrimido[4,5-d]pyrimidin-3-yl)-4-methylphenyl]-3-trifluoromethylbenzamide (35.0 mg, 73.6 mmol) and phenylenediamine (79.5 mg, 736 mmol) is stirred for 1 hour at 100° C. The mixture is cooled to room temperature and suspended in methanol. The precipitate is collected and washed with methanol to give N-{3-[7-(3-amino-phenylamino)-1-methyl-2-oxo-1,4-dihydro-2H-pyrimido[4,5-d]pyrimidin-3-yl]-4-methyl-phenyl}-3-trifluoromethyl-benzamide (34 mg, 84%) as a white solid; ¹H NMR 400 MHz (DMSO-d₆) δ 9.22 (s, 1H), 8.29 (s, 1H), 8.25 (d, 1H), 8.10 (s, 1H), 7.95 (d, 1H), 7.78-7.76 (m, 2H), 7.62 (dd, 1H), 7.30 (d, 1H), 7.05 (d, 1H), 6.88 (d, 1H), 6.87 (s, 1H), 6.17 (dd, 1H), 4.92 (s, 2H), 4.67 (d, 1H), 4.49 (d, 1H), 3.33 (s, 3H), 2.12 (s, 3H); MS m/z 548.3 (M+1).

### Example 2

### N-[4-Methyl-3-(1-methyl-7-methylamino-2,4-dioxo-1,4-dihydro-2H-pyrimido[4,5-d]pyrimidin-3-yl)-phenyl]-3-trifluoromethyl-benzamide

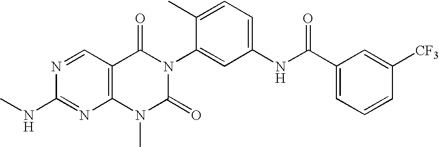

To a stirred solution of ethyl 4-chloro-2-methylsulfanyl-5-pyrimidinecarboxylate (4.50 g, 19.4 mmol) in MeOH is added 7 N NH₃ (13.9 mL) in MeOH at 0° C. and the mixture is stirred for 2 h at room temperature. The reaction mixture is diluted with EtOAc and washed with saturated NaHCO₃ solution. The organic layer is dried over MgSO₄, filtered and concentrated. The crude product is crystallized from the mixed solvent of EtOAc and hexanes to give 2.90 g (66%) of ethyl 4-amino-2-methylsulfanyl-5-pyrimidinecarboxylate as a white solid.

To a stirred solution of ethyl 4-amino-2-methylsulfanyl-5-pyrimidinecarboxylate (2.79 g, 13.1 mmol) is added 4 N NaOH (3.9 mL) and the mixture is stirred for 3 h at 60° C. The reaction mixture is concentrated to give 4-amino-2-methylsulfanyl-5-pyrimidinecarboxylate in a sodium salt form in quantitative yield.

To a solution of 4-amino-2-methylsulfanyl-5-pyrimidinecarboxylate in a sodium salt form (1.28 g, 6.2 mmol), N-(3-Amino-4-methyl-phenyl)-3-trifluoromethyl-benzamide (1.82 g, 6.2 mmol), and DIEA (3.22 mL, 18.5 mmol) in DMF is added HATU (2.82 g, 7.42 mmol), and the mixture is stirred for 1 h at room temperature. The reaction mixture is diluted with EtOAc and washed with 5% aqueous Na₂S₂O₃ solution, saturated aqueous NaHCO₃ solution, and brine. The organic layer is dried over MgSO₄ and concentrated in reduced pressure. The crude product is crystallized from MeOH to give 4-amino-2-methylsulfanyl-pyrimidine-5-carboxylic acid [2-methyl-5-(3-trifluoromethyl-benzoylamino)-phenyl]-amide (1.79 g, 61%) as a white solid.

To a stirred solution of 4-amino-2-methylsulfanyl-pyrimidine-5-carboxylic acid [2-methyl-5-(3-trifluoromethyl-benzoylamino)-phenyl]amide (286 mg, 0.62 mmol) and diisopropylethylamine (864 μL, 4.96 mmol) in dioxane (10 mL) is added a solution of triphosgene (184 mg, 0.62 mmol) in dioxane (2 mL) at 0° C., and the mixture is stirred for 12 h at 100° C. The reaction mixture is diluted with EtOAc (50 mL), and washed with saturated NaHCO₃ solution. The organic layer is dried over MgSO₄, filtered, concentrated under reduced pressure, and crystallized from MeOH to give N-[4-Methyl-3-(7-methylsulfanyl-2,4-dioxo-1,4-dihydro-2H-pyrimido[4,5-d]pyrimidin-3-yl)-phenyl]-3-trifluoromethyl-benzamide (166 mg, 55%) as a white crystalline solid.

To the suspension of NaH (60% dispersion in mineral oil, 19.7 mg, 0.49 mmol) in DMF is added N-[4-Methyl-3-(7-methylsulfanyl-2,4-dioxo-1,4-dihydro-2H-pyrimido[4,5-d]pyrimidin-3-yl)-phenyl]-3-trifluoromethyl-benzamide (218 mg, 0.45 mmol) at 0° C. When H₂ evolution has ceased, iodomethane (84 μl, 1.35 mmol) is added and the reaction mixture is stirred for 3 hours at room temperature. The mixture is diluted with ethyl acetate, and washed with 5% aqueous Na₂S₂O₃ solution to remove DMF. The organic layer is dried over MgSO₄ and concentrated under reduced pressure. The crude product is crystallized from MeOH to give N-[4-Methyl-3-(1-methyl-7-methylsulfanyl-2,4-dioxo-1,4-dihydro-2H-pyrimido[4,5-d]pyrimidin-3-yl)-phenyl]-3-trifluoromethyl-benzamide (184 mg, 82%) as a white solid.

To a stirred solution of N-[4-Methyl-3-(1-methyl-7-methylsulfanyl-2,4-dioxo-1,4-dihydro-2H-pyrimido[4,5-d]pyrimidin-3-yl)-phenyl]-3-trifluoromethyl-benzamide (184 mg, 0.37 mmol) in the mixed solvent of DMF (4 mL) and chloroform (4 mL) is added in-chloroperoxybenzoic acid (77% max., 97 mg, 44 mmol) and the mixture is stirred for 1 h at room temperature. The mixture is diluted with chloroform, and washed with 5% aqueous Na₂S₂O₃ solution and saturated NaHCO₃ solution. The organic layer is dried over MgSO₄ and concentrated under reduced pressure to give N-[3-(7-Methanesulfinyl-1-methyl-2,4-dioxo-1,4-dihydro-2H-pyrimido[4,5-d]pyrimidin-3-yl)-4-methyl-phenyl]-3-trifluoromethyl-benzamide (167 mg, 88%).

N-[3-(7-Methanesulfinyl-1-methyl-2,4-dioxo-1,4-dihydro-2H-pyrimido[4,5-d]pyrimidin-3-yl)-4-methyl-phenyl]-3-trifluoromethyl-benzamide (30 mg, 58 μmol) is dissolved in 2 M methylamine solution (1 mL) in THF and the mixture is stirred for 1 h at 60° C. The reaction mixture is concentrated, dissolved in DMSO, and purified by preparative LCMS to give N-[4-Methyl-3-(1-methyl-7-methylamino-2,4-dioxo-1,4-dihydro-2H-pyrimido[4,5-d]pyrimidin-3-yl)-phenyl]-3-trifluoromethyl-benzamide (20 mg, 71%); ¹H NMR 400 MHz (DMSO-d₆) δ 10.70 (s, 1H), 8.95 (s, 0.33H), 8.85 (s, 0.66H), 8.39 (m, 3H), 8.11 (d, 1H), 7.93 (t, 1H), 7.84 (m, 2H), 7.49 (d, 1H), 3.65 (d, 2H), 3.58 (s, 1H), 3.08 (m, 3H), 2.17 (s, 3H); MS m/z 485.3 (M+1).

By repeating the procedures described in the above examples, using appropriate starting materials, the following compounds of Formula I, as identified in Table 1, are obtained.

TABLE 1

| | | Physical Data |
|---|---|---|
| | | ¹H NMR 400 MHz |
| Compound | | (DMSO-d₆) and/or MS |
| Number | Structure | (m/z) |
| 3 | 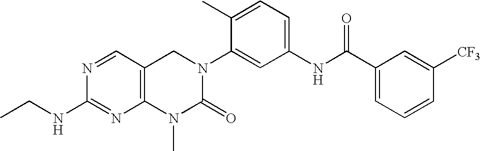 | MS m/z 485.4 (M + 1) |
| 4 | 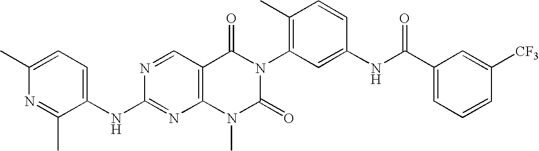 | MS m/z 576.4 (M + 1) |
| 5 | 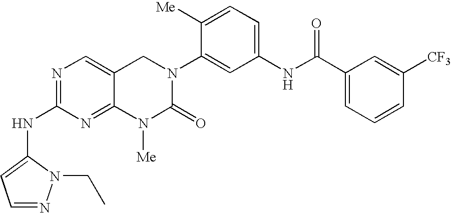 | MS m/z 551.1 (M + 1). |
| 6 | 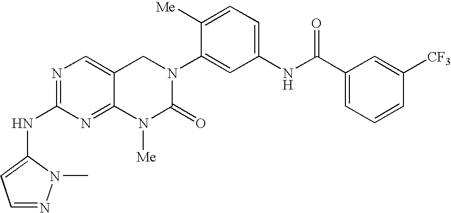 | MS m/z 537.1 (M + 1). |
| 7 | 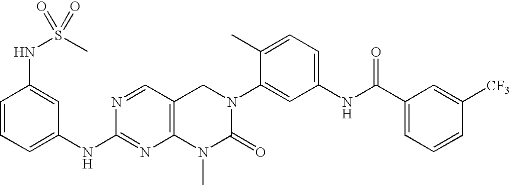 | ¹H NMR 400 MHz (DMSO-d₆) δ 9.64 (s, 1H), 9.60 (d, 1H), 8.29 (s, 1H), 8.21 (d, 1H), 8.14 (s, 1H), 7.95 (d, 1H), 7.78 (m, 3H), 7.63 (d, 1H), 7.42 (d, 1H), 7.31 (d, 1H), 7.20 (t, 1H), 6.77 (d, 1H), 4.68 (d, 1H), 4.53 (d, 1H), 3.35 (s, 3H), 2.97 (s, 3H), 2.13 (s, 3H); MS m/z 626.3 (M + 1). |
| 8 | 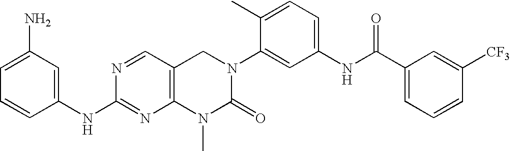 | ¹H NMR 400 MHz (DMSO-d₆) δ 9.22 (s, 1H), 8.29 (s, 1H), 8.25 (d, 1H), 8.10 (s, 1H), 7.95 (d, 1H), 7.78-7.76 (m, 2H), 7.62 (dd, 1H), 7.30 (d, 1H), 7.05 (d, 1H), 6.88 (d, 1H), 6.87 (s, 1H), 6.17 (dd, 1H), 4.92 (s, 2H), 4.67 (d, 1H), 4.49 (d, 1H), 3.33 (s, 3H), 2.12 (s, 3H); MS m/z 548.3 (M + 1). |
| 9 | 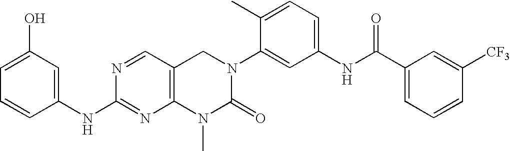 | ¹H NMR 400 MHz (DMSO-d₆) δ 10.51 (s, 1H), 9.43 (s, 1H), 9.22 (s, 1H), 8.29 (s, 1H), 8.25 (d, 1H), 8.12 (s, 1H), 7.96 (d, 1H), 7.80-7.77 (m, 2H), 7.62 (dd, 1H), 7.29-7.31 (m, 2H), 7.13 (d, 1H), 7.02 (t, 1H), 6.34 (d, 1H), 4.68 (d, 1H), 4.51 (d, 1H), 3.34 (s, 3H), 2.12 (s, 3H); MS m/z 549.2 (M + 1). |

### Assays

Using a feeder cell dependent mouse ES cell line (which is engineered with a Oct4-GFP reporter construct and expresses GFP in the undifferentiated, pluripotent state), compounds are screened for their ability to maintain the undifferentiated state of ES cells without feeder cells and LIF. Compounds of the invention maintain mouse ES cells in the undifferentiated states for greater than 10 passages without the need for LIF and feeder layers. Pluripotent ES cells express Oct4, Nanog, ALP, SSEA-1 and form compact colonies. Differentiations are indicated by the presence of loose colonies and flat and/or cobble-stone like cells. The mouse ES cells expanded by the compound of the invention retain multiple markers of pluripotent cells, including Oct-4, nanog, SSEA-1 and ALP and can differentiate into functional neuronal and cardiac cells in vitro and contribute to healthy chimeric mice in vivo. It is also found that compounds of the invention do not activate Wnt pathway by the described TOPflash reporter assay and do not active JAK-STAT pathway by western blotting.

Maintenance of Mouse Embryonic Stem (mES) Cell Self-Renewal

Mouse ES cells are maintained with feeder layer cells in GM on gelatin-coated plates. Mouse ES cells are passaged every three days using 0.05% trypsin-EDTA (0.5 ml/well). The optimal split ratio is 1:6.

Materials used for ES cell maintenance, and examples 4 & 5, infra, include: Oct4-GFP mES cells (feeder layer dependent cells); mES R1 cells (feeder layer independent cells); DMEM (GIBCO, 11965-084); Kouckout DMEM (KO DMEM) (GIBCO, 10829-018); DMEM/F12 (GIBCO, 11330-032); Fetal Bovine Serum (FBS) (GIBCO, 26140-079); Knockout Serum Replacer (KO-SR), (GIBCO, 10828-028); B-27 Serum-free Supplement (50X), (GIBCO017504-044); N-2 Supplement (100X) (GIBCO, 17502-048); LIF (10⁶ units) (Chemicon, ESG1106); L-Glutamine (GIBCO, 25030-081); Non-essential amino acids (GIBCO, 11140-050); 2-Mercaptoethanol (1000X), (GIBCO, 21985-023); 0.05% Trypsin-EDTA (GIBCO, 25300-054); 0.1% gelatin solution (Stemcell tech., 07903); Basal medium (BM): KO DMEM, 15% KO-SR, 1X L-glutamine, 1X non-essential amino acid, 1×2-mercaptoethanol; and Growth medium (GM): Basal medium+10³ unit LIF.

### Screening to Identify Compounds of the Invention:

The 384 well plates are coated with 0.1% gelatin solution at 37° C. overnight. The gelatin solution is removed by aspiration. Oct4-GFP mouse ES (feeder layer dependent) cells are plated on gelatin-coated plates at 1000 cells/50 μl GM/well. After overnight incubation, the medium is changed to BM and 5 μM of compound is added to each well. After 3 days incubation, the medium is replaced and compound is added again. After a further 3 days, the cells are fixed and assayed using a fluorometric imaging plate reader system (FLIPR). The wells in which the cells kept the GFP expression are picked as primary hits. The primary hits are further confirmed with the colony morphology of mouse ES cells. Using this method, compounds of the invention are identified that maintain the mouse ES cell self-renewal under feeder layer-free condition.

### Example 3

### Mouse Es Cells Keep Pluripotency Under Differentiation Medium (DM)

DM induced by retinoic acid (RA): BM+0.3 μM RA, DM induced by FBS: DMEM, 20% FBS. Ninety-six well plates are coated with 0.1% gelatin solution at 37° C. overnight. The gelatin solution is removed by aspiration. Mouse embryonic stem cells are plated on gelatin-coated plates at 10⁴ cells/50 μl GM/well. After overnight incubation, the medium is changed to DM and 3 μM of a compound of the invention is added to each well. After 3 days incubation, the medium is replaced with fresh medium and compound. After a further 3 days, the cells are fixed and assayed with pluripotent markers expression and colony morphology. An effective concentration is measured by the maintenance of GFP expression and colony morphology. A list of effective concentrations for various compounds of the invention is disclosed in table 3, infra

### Example 4

### Feeder Layer-Free Multiple Passages Culture Condition

Six well plates are coated with 1 ml of 0.1% gelatin per well and incubate at 37° C. overnight. After removal of gelatin solution, mouse ES cells are plated at 2×10⁵ cells/2 ml culture medium per well. Cells are passaged every 3 days using 0.05% Trypsin-EDTA (0.5 ml/well). The optimal split ratio is dependent on different culture medium (table 2). Table 2 shows examples of different feeder layer-free culture conditions where the compound of the invention is N-{4-Methyl-3-[1-methyl-7-(2-methyl-2H-pyrazol-3-ylamino)-2-oxo-1,4-dihydro-2H-pyrimido[4,5-d]pyrimidin-3-yl]-phenyl}-3-trifluoromethyl-benzamide (compound 213, table 1).

TABLE 2
Different feeder layer-free culture conditions.

| | | Optimal |
|---|---|---|
| | | split |
| | Culture Medium | ratio |
| Serum-containing | Basal medium + 3 μM compound of the | 1:6 |
| condition | invention | |
| Serum-free condition | DMEM/F12, 1X N2 supplement, | 1:3 |
| | 1X B27 supplement, 1X L-glutamine, | |
| | 1X-non essential amino acid, 1X | |
| | 2-Mercaptoethanol, 1 μM compound | |
| | of the invention | |
| Optimized Serum-free | DMEM/F12, 1X N2 supplement, | 1:4 |
| condition-N2B27 | 1X B27 supplement, 1X L-glutamine, | |
| | 1X-non essential amino acid, 1X | |
| | 2-Mercaptoethanol, 10³ LIF, | |
| | 300 nM compound of the invention | |
| Optimized Serum-free | DMEM/F12, 1X N2 supplement, | 1:3 |
| condition-N2 | 1X L-glutamine, 1X-non essential | |
| | amino acid, 1X 2-Mercaptoethanol, 10³ | |
| | LIF, 300 nM compound of the invention | |

TABLE 3

| | | Effective |
|---|---|---|
| Compound Structure | Compound Name | Concentration |
| 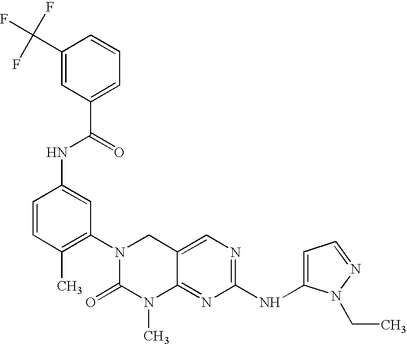 | N-{3-[7-(2-Ethyl-2H-pyrazol- 3-ylamino)-1-methyl-2-oxo- 1,4-dihydro-2H-pyrimido[4,5- d]pyrimidin-3-yl]-4-methyl- phenyl}-3-trifluoromethyl- benzamide | 2 μM |
| 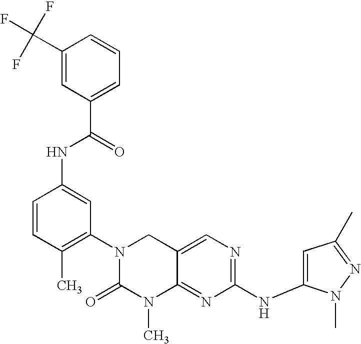 | N-{3-[7-(2,5-Dimethyl-2H- pyrazol-3-ylamino)-1-methyl- 2-oxo-1,4-dihydro-2H- pyrimido[4,5-d]pyrimidin-3- yl]-4-methyl-phenyl}-3- trifluoromethyl-benzamide | 1 μM |
| 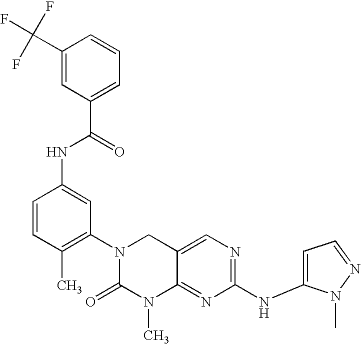 | N-{4-Methyl-3-[1-methyl-7- (2-methyl-2H-pyrazol-3- ylamino)-2-oxo-1,4-dihydro- 2H-pyrimido[4,5- d]pyrimidin-3-yl]-phenyl}-3- trifluoromethyl-benzamide | 1 μM |
| 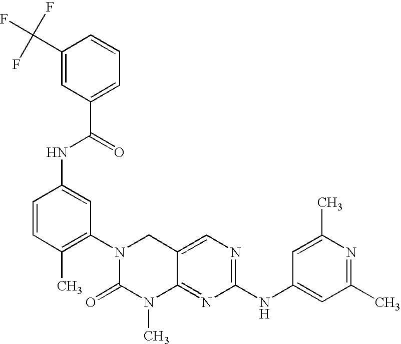 | N-{3-[7-(2,6-Dimethyl- pyridin-4-ylamino)-1-methyl- 2-oxo-1,4-dihydro-2H- pyrimido[4,5-d]pyrimidin-3- yl]-4-methyl-phenyl}-3- trifluoromethyl-benzamide | 5 μM |
| 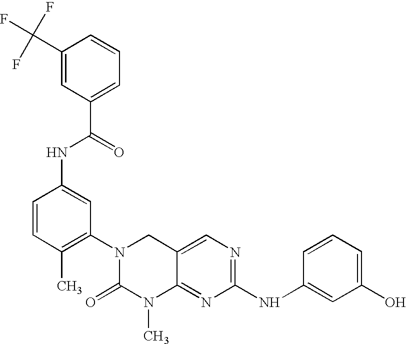 | N-{3-[7-(3-Hydroxy- phenylamino)-1-methyl-2- oxo-1,4-dihydro-2H- pyrimido[4,5-d]pyrimidin-3- yl]-4-methyl-phenyl}-3- trifluoromethyl-benzamide | 1 μM |
| 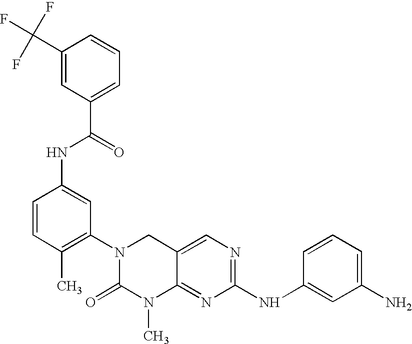 | N-{3-[7-(3-Amino- phenylamino)-1-methyl-2- oxo-1,4-dihydro-2H- pyrimido[4,5-d]pyrimidin-3- yl]-4-methyl-phenyl}-3- trifluoromethyl-benzamide | 2 μM |
| 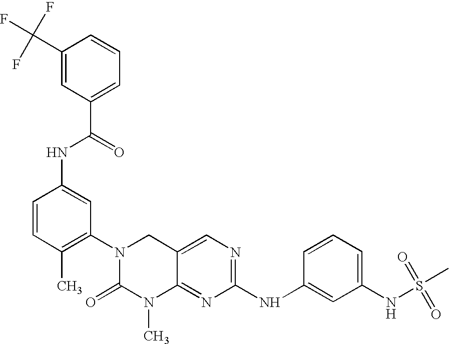 | N-{3-[7-(3- Methanesulfonylamino- phenylamino)-1-methyl-2- oxo-1,4-dihydro-2H- pyrimido[4,5-d]pyrimidin-3- yl]-4-methyl-phenyl}-3- trifluoromethyl-benzamide | 2 μM |
| 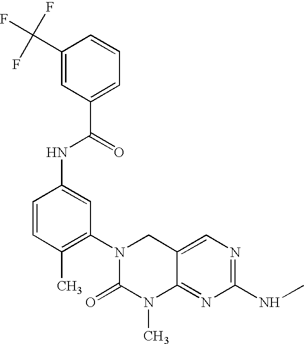 | N-(4-Methyl-3-(1-methyl-7- methylamino-2-oxo-1,4- dihydro-2H-pyrimido[4,5- d]pyrimidin-3 -yl)-phenyl]-3- trifluoromethyl-benzamide | 3 μM |
| 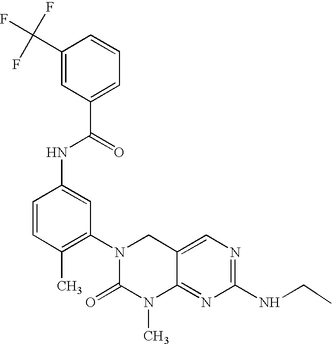 | N-[3-(7-Ethylamino-1- methyl-2-oxo-1,4-dihydro- 2H-pyrimido[4,5- d]pyrimidin-3-yl)-4-methyl- phenyl]-3-trifluoromethyl- benzamide | 10 μM |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A method of maintaining pluripotent stem cells, comprising the steps of growing the cells in: a) a basal medium; and b) a compound of Formula I: 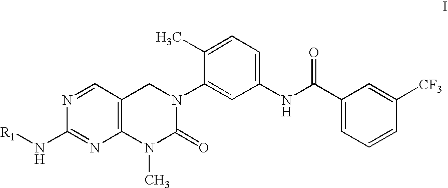
in which:
R₁ is selected from hydrogen, C₁₋₆alkyl, C₂₋₆alkenyl, C₆₋₁₀aryl-C₀₋₄alkyl, C₅₋₁₀heteroaryl-C₀₋₄alkyl, C₃₋₁₀cycloalkyl-C₀₋₄alkyl and C₃₋₁₀heterocycloalkyl-C₀₋₄alkyl; wherein any alkyl or alkenyl of R₁ is optionally substituted by one to three radicals independently selected from halo, hydroxy, C₁₋₆alkyl and —NR₂R₃; wherein any aryl, heteroaryl, cycloalkyl or heterocycloalkyl of R₁ is optionally substituted by one to three radicals selected from halo, hydroxy, cyano, C₁₋₆alkyl, C₁₋₆alkoxy, C₂₋₆alkenyl, halo-substituted-alkyl, halo-substituted-alkoxy, —XNR₂R₃, —XOXNR₂R₃, —XNR₂S(O)₀₋₂R₃, —XC(O)NR₂R₃, —XNR₂C(O)XOR₂, —XNR₂C(O)NR₂R₃, —XNR₂XNR₂R₃, —XC(O)NR₂XNR₂R₃, —XNR₂XOR₂, —XOR₂, —XNR₂C(═NR₂)NR₂R₃, —XS(O)₀₋₂R₄, —XNR₂C(O)R₂, —XNR₂C(O)XNR₂R₃, —XNR₂C(O)R₄, —XC(O)R₄, —XR₄, —XC(O)OR₃ and —XS(O)₀₋₂NR₂R₃; wherein X is a bond or C₁₋₄alkylene; R₂ and R₃ are independently selected from hydrogen, C₁₋₆alkyl and C₃₋₁₂cycloalkyl; and R₄ is C₃₋₁₀heterocycloalkyl optionally substituted with 1 to 3 radicals selected from C₁₋₆alkyl, —XNR₂R₃, —XNR₂XNR₂R₂, XNR₂XOR₂ and —XOR₂; wherein X, R₂ and R₃ are as described above; and the pharmaceutically acceptable salts, hydrates, solvates and isomers thereof.

2. The method of claim 1 wherein the cells are mammalian cells.

3. The method of claim 1 wherein the cells are human embryonic stem cells.

4. The compound of claim 4 in which R₁ is selected from hydrogen, methyl, ethyl, isopropyl, cyclopropyl, cyclobutyl, cyclopentyl, cyclohexyl, pyrimidinyl, 3-hydroxy-1-methyl-propyl hydroxy-ethyl, phenyl, morpholino, benzyl, [1,2,4]triazol-4-yl, allyl, 2-(2-oxo-pyrrolidin-1-yl)-ethyl, piperazinyl-ethyl, piperazinyl-propyl, thiazolyl, oxazolyl, pyridinyl, pyrazolyl, piperidinyl, thiazolyl, ethyl-pyrrolidinyl-methyl, morpholino-propyl, dimethyl-amino-propyl, diethyl-amino-propyl, diethyl-amino-butyl, ethoxy-carbonyl-methyl and [1,2,4]triazin-3-yl, [1,3,4]thiadiazolyl; wherein any aryl, heteroaryl, cycloalkyl or heterocycloalkyl is optionally substituted with 1 to 3 radicals independently selected from methyl, ethyl, cyano, hydroxy, methoxy, amino-carbonyl-amino, hydroxy-methyl, methyl-piperazinyl, methyl-piperazinyl-carbonyl, ethyl-piperazinyl, methyl-piperazinyl-methyl, morpholino-sulfonyl, methyl-piperazinyl-sulfonyl, methyl-piperazinyl-carbonyl-amino, methyl-sulfonyl-amino, amino-carbonyl, amino-sulfonyl, hydroxy-ethyl, hydroxy-methyl-carbonyl-amino, formyl-amino, dimethyl-amino, dimethyl-amino-methyl, dimethyl-amino-ethyl, isopropyl-amino-ethyl, carboxy, amino-ethyl-amino, methyl-amino-ethyl, morpholino-ethyl, morpholino-methyl, amino-ethyl, imidazolyl-propyl, piperazinyl-ethyl, piperazinyl, trifluoromethyl, diethyl-amino-ethyl, fluoro, morpholino, dimethyl-amino-ethyl-amino-carbonyl, diethyl-amino-ethoxy, 2-amino-propionylamino, dimethyl-amino-pyrrolidinyl, (2-dimethylamino-ethyl)-methyl-amino, 2-dimethylamino-1-methyl-ethoxy and diethyl-amino.

5. The compound of claim 4 selected from: N-{3-[7-(2-Ethyl-2H-pyrazol-3-ylamino)-1-methyl-2-oxo-1,4-dihydro-2H-pyrimido[4,5-d]-pyrimidin-3-yl]-4-methyl-phenyl}-3-trifluoromethyl-benzamide; N-{4-Methyl-3-[1-methyl-7-(2-methyl-2H-pyrazol-3-ylamino)-2-oxo-1,4-dihydro-2H-pyrimido[4,5-d]pyrimidin-3-yl]-phenyl}-3-trifluoromethyl-benzamide; N-{3-[7-(2,6-Dimethyl-pyridin-4-ylamino)-1-methyl-2-oxo-1,4-dihydro-2H-pyrimido[4,5-d]pyrimidin-3-yl]-4-methyl-phenyl-3-trifluoromethyl-benzamide; N-{3-[7-(3-Hydroxy-phenylamino)-1-methyl-2-oxo-1,4-dihydro-2H-pyrimido[4,5-d]pyrimidin-3-yl]-4-methyl-phenyl}-3-trifluoromethyl-benzamide; N-{3-[7-(3-Amino-phenylamino)-1-methyl-2-oxo-1,4-dihydro-2H-pyrimido[4,5-d]pyrimidin-3-yl]-4-methyl-phenyl}-3-trifluoromethyl-benzamide; N-{3-[7-(3-Methanesulfonylamino-phenylamino)-1-methyl-2-oxo-1,4-dihydro-2H-pyrimido[4,5-d]pyrimidin-3-yl]-4-methyl-phenyl}-3-trifluoromethyl-benzamide; N-{3-[7-(2,5-Dimethyl-2H-pyrazol-3-ylamino)-1-methyl-2-oxo-1,4-dihydro-2H-pyrimido[4,5-d]pyrimidin-3-yl]-4-methyl-phenyl}-3-trifluoromethyl-benzamide; N-[4-Methyl-3-(1-methyl-7-methylamino-2-oxo-1,4-dihydro-2H-pyrimido[4,5-d]pyrimidin-3-yl)-phenyl]-3-trifluoromethyl-benzamide; and N-[3-(7-Ethylamino-1-methyl-2-oxo-1,4-dihydro-2H-pyrimido[4,5-d]pyrimidin-3-yl)-4-methyl-phenyl]-3-trifluoromethyl-benzamide.
